(19) United States
(12) Patent Application Publication
(10) Pub. No.: US 2026/0166230 A1
(43) Pub. Date: Jun. 18, 2026

(54) INJECTABLE MATERIAL HYDRATING DEVICES AND RELATED METHODS

(71) Applicant: Merit Medical Systems, Inc., South Jordan, UT (US)

(72) Inventors: Michael Dean Haslam, Sandy, UT (US); Nicole Willardson, Lehi, UT (US); Richard P. Jenkins, Bluffdale, UT (US)

(21) Appl. No.: 19/439,129

(22) Filed: Jan. 2, 2026

Related U.S. Application Data
(63) Continuation of application No. 18/493,446, filed on Oct. 24, 2023.
(60) Provisional application No. 63/380,893, filed on Oct. 25, 2022.
(60) Provisional application No. 63/486,598, filed on Feb. 23, 2023.

(51) Int. Cl. A61M 5/31; A61M 5/315

(52) U.S. Cl. CPC A61M 5/3129; A61M 5/31505; A61M 2005/3131; A61M 2005/31506; A61M 2205/0222; A61M 2207/00

(57) ABSTRACT

A syringe system including a plunger member that includes a plunger rod defining a proximal end and a distal end, a plunger tip disposed at the distal end, and a seal member coupled to the plunger tip. The plunger tip is configured to prevent movement of the plunger rod relative to the seal member.

### RELATED APPLICATIONS

This application is a Continuation of U.S. patent application Ser. No. 18/493,446, filed on Oct. 24, 2023 and titled “Injectable Material Hydrating Devices and Related Methods”, which claims priority to U.S. Provisional Application No. 63/380,893, filed on Oct. 25, 2022 and titled, “Injectable Material Hydrating Devices and Related Methods” and U.S. Provisional Application No. 63/486,598 , filed on Feb. 23, 2023 and titled, “Injectable Material Hydrating Devices and Related Methods,” all of which are hereby incorporated by reference in their entireties.

### TECHNICAL FIELD

The present disclosure relates generally to syringe systems comprising barrels including lubricated portions and non-lubricated portions. More specifically, in some embodiments, the present disclosure relates to barrels including non-lubricated portions configured to be loaded with injectable materials and methods of lubricating portions of the barrels. This disclosure also relates to various embodiments of plunger members, including plunger members having plunger tips configured to couple with a seal member. Related methods are also disclosed.

### BRIEF DESCRIPTION OF THE DRAWINGS

The embodiments disclosed herein will become more fully apparent from the following description and appended claims, taken in conjunction with the accompanying drawings. While various aspects of the embodiments are presented in drawings, the drawings depict only typical embodiments, which will be described with additional specificity and detail through use of the accompanying drawings in which:

FIG. 1 is a perspective view of an embodiment of a syringe system.

FIG. 2 is a cross-sectional side view of the syringe system of FIG. 1, wherein a non-lubricated portion of a barrel is loaded with an injectable material.

FIG. 3 is a perspective view of an embodiment of a plunger of the syringe system of FIG. 1.

FIG. 4 is a perspective view of an embodiment of a plunger rod of the plunger of FIG. 3.

FIG. 5A is a perspective view of an embodiment of a plunger tip of the plunger of FIG. 3.

FIG. 5B is a cross-sectional side view of plunger tip of FIG. 5A.

FIG. 6A is a perspective view of an embodiment of a seal member of the plunger of FIG. 3.

FIG. 6B is a cross-sectional side view of the seal member of FIG. 6A.

FIG. 7A is a cross-sectional side view of the syringe system of FIG. 1, wherein the injectable material is disposed in non-lubricated portion of a barrel.

FIG. 7B is a cross-sectional side view of the syringe system of FIG. 1, wherein the injectable material, plunger tip, and seal member are disposed in the barrel.

FIG. 7C is a cross-sectional side view of the syringe system of FIG. 1, wherein a lubricant is being applied to a lubricated portion of an interior surface of a first wall of the barrel.

FIG. 7D is a cross-sectional side view of the syringe system of FIG. 1, wherein the injectable material, plunger tip, and seal member are disposed in the barrel, the lubricant coats the lubricated portion of the interior surface of the first wall of the barrel, and the plunger rod is coupled to the plunger tip.

FIG. 8 is a cross-sectional side view of another embodiment of a syringe system.

FIG. 9 is a perspective view of a plunger of the syringe system of FIG. 8.

FIG. 10A is a perspective view of an embodiment of an injectable material container.

FIG. 10B is a cross-sectional side view of the injectable material container of FIG. 10A.

FIG. 11A is a rear perspective view of an embodiment of a plunger tip.

FIG. 11B is a cross-sectional side view of plunger tip of FIG. 11A.

FIG. 11C is a front perspective view of the plunger tip of FIG. 11A.

FIG. 12 is a perspective view of a plunger for use with the plunger tip of FIG. 11A.

### DETAILED DESCRIPTION

The various embodiments disclosed herein generally relate to syringe systems comprising barrels including lubricated portions and non-lubricated portions. In some embodiments, the non-lubricated portions are configured to be loaded with injectable materials. In certain embodiments, the injectable materials may comprise polyvinyl alcohol (PVA). In various embodiments, the injectable materials may comprise a substance including gelatin foam (e.g., GELFOAM®). Also disclosed herein are methods of lubricating portions of barrels of syringe systems.

Embodiments may be understood by reference to the drawings, wherein like parts are designated by like numerals throughout. It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail. While the various aspects of the embodiments are presented in drawings, the drawings are not necessarily drawn to scale unless specifically indicated.

The term “silicone” refers to polymers that include repeating units of siloxane (e.g., poly-siloxane ((R₂SiO)ₙ)). As used herein, the terms silicone and poly-siloxane may be used interchangeably.

The term “shelf stable” refers to syringe systems, wherein any components of the syringe system, the injection material, and/or an interaction between the injection material and the syringe system (i.e., an interaction in a pre-loaded syringe system) are such that after a given timeframe (e.g., six months, one year, two years, three years, etc.) the syringe system and the injection material are still suitable for their intended purpose. For example, when the injection material comprises an embolic agent (e.g., a microparticle or a microsphere), agglomeration of the embolic agent may render the pre-loaded syringe system unsuitable for its intended purpose. For example, agglomeration of more than about 10% of the embolic agent may render the pre-loaded syringe unsuitable for its intended purpose. Likewise, adhesion of the injection material to one or more components of the syringe system (e.g., a barrel) may render the pre-loaded syringe unsuitable for its intended purpose.

The term “sterilization compatible” refers to components or materials configured to be sterilized without rendering the components or materials unsuitable for their intended purpose. If a component or material is configured for sterilization by at least one method of sterilization without being rendered unsuitable for its intended purpose, then the material is “sterilization compatible.” For example, a polymeric barrel may deform when autoclaved at temperatures sufficient to sterilize the barrel, rendering the barrel unsuitable for maintaining a seal with a seal member of a plunger. However, if the same polymeric barrel may be sterilized by another sterilization technique (e.g., by irradiation) and maintain suitability for its intended purpose, then the polymeric material is “sterilization compatible.”

The term “irradiation compatible” refers to components or materials configured to be sterilized by irradiation without rendering the components or materials unsuitable for their intended purpose. For example, a plunger or an interior surface of a barrel may comprise a material (e.g., a lubricant) that upon irradiation changes in physical properties such that the syringe is unsuitable for its intended purpose. For example, irradiation may alter certain materials such that a syringe using those materials would have an unacceptably high initial peak force required to start movement of a plunger. Or, in another example, irradiation may alter certain materials such that a syringe using those materials would have an unacceptably non-uniform force required for travel of the plunger over the length of the barrel or an unacceptably high force required for travel of the plunger over the length of the barrel.

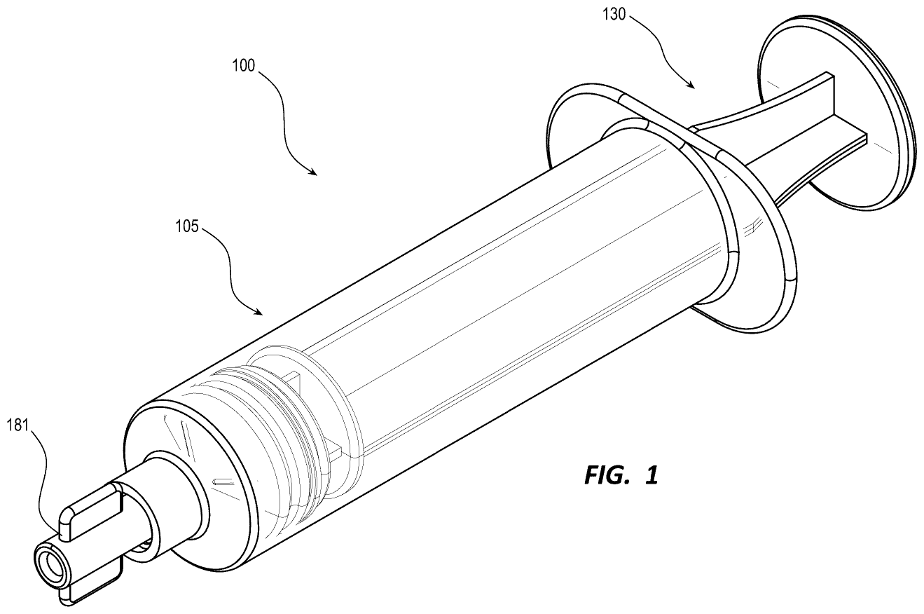

FIG. 1 is a perspective view of an embodiment of a syringe system 100. As illustrated, the syringe system 100 can include a syringe body 105, a plunger member 130, and a non-vented cap 181. In a ready-to-use state, the plunger member is at least partially disposed within the syringe body 105 and the non-vented cap 181 is coupled to a distal end of the syringe body 105. Additionally, in a ready-to-use state, a portion of the syringe body 105 can be filled with an injectable material, as will be discussed below.

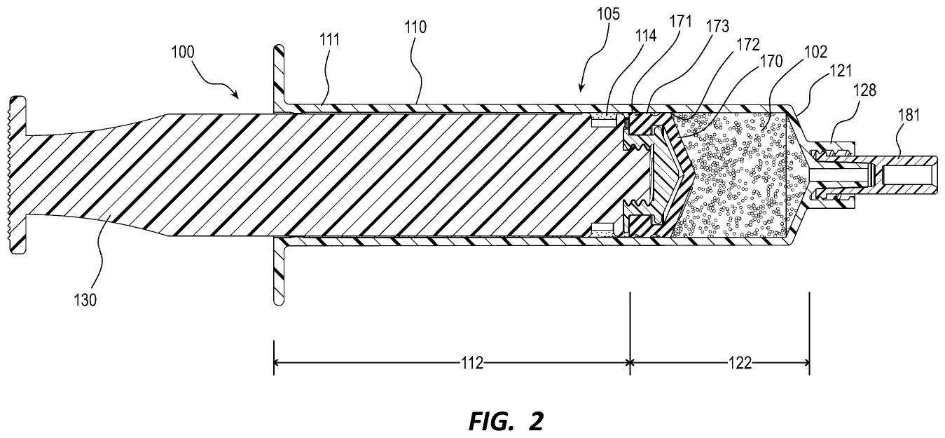

FIG. 2 is a cross-sectional side view of the syringe system 100. As depicted, the syringe system 100 can comprise the syringe body 105 and the plunger member 130. In some embodiments, the plunger member 130 may be configured to be disposed and/or displaceable within at least a portion of a barrel 110 of the syringe body 105. The syringe system 100 can also comprise an inlet/outlet port 128 disposed at a distal end of the syringe body 105. The non-vented cap 181 can be coupled to the inlet/outlet port 128. In some embodiments, the non-vented cap 181 is configured to prevent outflow of any material contained within the barrel 110, such as an injectable material. In various embodiments, the barrel 110 or the syringe body 105 may be formed from a material selected from at least one of a polymer, a glass, and/or a metal. In various embodiments, the syringe body 105 and/or the barrel 110 may comprise a polymeric material such as, for example, a polycarbonate, a polypropylene, and/or a cyclo-olefin polymer or copolymer. Barrels 110 and/or syringe bodies 105 formed from other suitable materials are also within the scope of this disclosure.

As illustrated, the barrel 110 can comprise a proximal or lubricated portion 112 and a distal or non-lubricated portion 122, wherein the non-lubricated portion 122 may be disposed distal of the lubricated portion 112. In some embodiments, at least a portion of an interior surface of a first wall 111 of the barrel 110 disposed in the lubricated portion 112 may be at least partially coated with a lubricant or lubricant coating 114. In contrast to the lubricated portion 112, at least a portion of the interior surface of the first wall 111 and an interior surface of a second wall 121 of the barrel 110 disposed in the non-lubricated portion 122 may lack a lubricant or lubricant coating. The lubricant 114 may aid in the displacement of the plunger member 130 within at least a portion of the barrel 110 of the syringe system 100 (i.e., the lubricated portion 112). For example, the plunger member 130 may be more easily displaced within the lubricated portion 112 of the barrel 110 than within the non-lubricated portion 122 of the barrel 110.

As illustrated, at least a portion of the barrel 110 (i.e., the non-lubricated portion 122) may be configured to receive or be loaded with an injectable material 102. For example, the injectable material 102 may be disposed within the non-lubricated portion 122. In some embodiments, the injectable material 102 may not be compatible with the lubricant 114. For example, the injectable material 102 may be at least partially formed from a material that may become contaminated, or become unsuitable for its intended purpose, upon contact with the lubricant 114. As such, in certain embodiments, the syringe system 100 may be configured such that the injectable material 102 may be disposed in the barrel 110 such that the lubricant 114 does not, or does not substantially, come into contact with the injectable material 102 during shipment, storage, and/or sterilization.

The syringe system 100 can comprise a pre-loaded syringe system. For example, as illustrated in FIG. 2, the syringe system 100 may be at least partially pre-loaded with the injectable material 102. In some embodiments, the pre-loaded syringe system 100 can comprise the barrel 110, wherein at least a portion of the barrel 110 is loaded with the injectable material 102 and/or the plunger member 130. In various embodiments, the pre-loaded syringe system 100 may be configured to be shelf stable for at least six months. In various other embodiments, the pre-loaded syringe system 100 may be configured to be shelf stable for at least one year, for at least two years, or for at least three years. Pre-loaded syringe systems 100 configured to be shelf stable for other suitable periods of time are also within the scope of this disclosure.

In various embodiments, the injectable material 102 may comprise PVA or be at least partially formed from PVA, while the lubricant 114 may comprise silicone or be at least partially formed from silicone. In various other embodiments, the injectable material 102 may comprise a substance including gelatin foam or be at least partially formed from a substance including gelatin foam. Disclosure of PVA herein may be analogously applied to other substances, for example, substances including gelatin foam. Materials comprising PVA or gelatin foam may become contaminated upon contact with or exposure to silicone. For example, PVA or gelatin foam may not be compatible with silicone. Accordingly, it may be desirable to provide a barrel 110 including both a lubricated portion 112 and a non-lubricated portion 122. The lubricated portion 112 may facilitate displacement or movement of the plunger member 130 within at least a portion of the barrel 110. Additionally, the non-lubricated portion 122 may be loaded with the injectable material 102 and provide a location or position within the syringe system 100 wherein the injectable material 102 may avoid or substantially avoid contact with or exposure to the lubricant 114 (e.g., until the injectable material 102 has been hydrated).

In certain embodiments, the injectable material 102 may be dry. In certain other embodiments, the injectable material 102 may be suspended in a liquid, such as, for example, a carrier liquid comprising saline. In some embodiments, the injectable material 102 may be dry during shipment and/or storage of the syringe system 100 or the barrel 110. In some other embodiments, the injectable material 102 may be suspended in a liquid or hydrated during shipment and/or storage of the syringe system 100 or the barrel 110.

In some embodiments, the injectable material 102 may be selected from at least one of embolic agents, polymeric microparticles, polymeric microspheres, etc. In some embodiments, the injectable material 102 may be an embolic agent comprising a microparticle and/or a microsphere. Examples of microparticles include, but are not limited to, PVA microparticles (e.g., BEARING™ non-spherical PVA microparticles) and/or gelatin foam microparticles (e.g., GELFOAM® microparticles). Examples of microspheres include, but are not limited to, trisacryl cross-linked with gelatin microspheres (e.g., EMBOSPHERE® microspheres), sodium acrylate vinyl alcohol copolymer microspheres (e.g., HEPASPHERE® microspheres), PVA-based hydrogels (e.g., DC BEAD® microspheres), and/or gelatin foam microspheres (e.g., GELFOAM® microspheres).

As described above, the injectable material 102 may comprise PVA or gelatin foam. In certain other embodiments, the injectable material 102 may comprise an embolic agent, wherein the embolic agent may comprise PVA or gelatin foam. An injection material or an embolic agent comprising PVA may include PVA monomers, such as PVA polymers and copolymers.

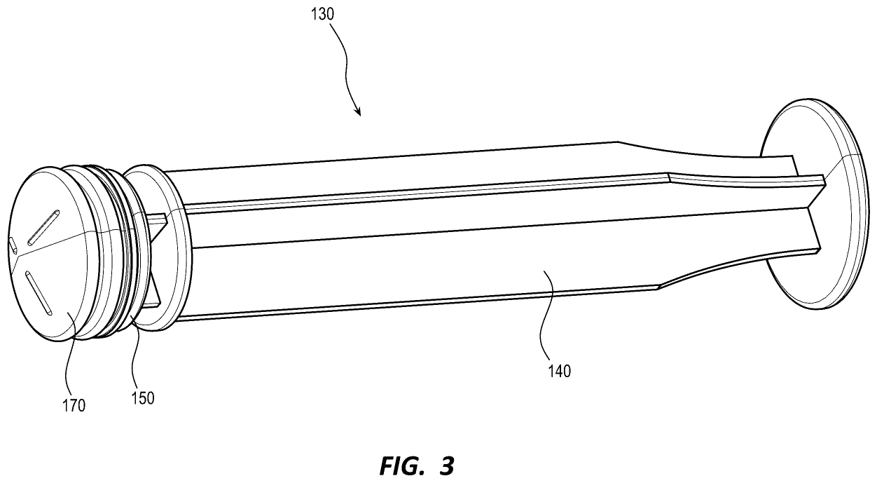

FIG. 3 is a perspective view of an embodiment of the plunger member 130. As shown in FIG. 3, the plunger member 130 can comprise a plunger rod 140, a plunger tip 150 coupled to a distal end of the plunger rod 140, and a seal member 170 coupled to the plunger tip 150 wherein the plunger tip 150 is disposed between the plunger rod 140 and the seal member 170. In some embodiments of the plunger member 130, a plunger rod 440 and a plunger tip 450 may replace the plunger rod 140 and the plunger tip 150. Unless otherwise noted below, the plunger rod 440 and the plunger tip 450 may include any aspect of the plunger rod 140 and the plunger tip 150 in the plunger member 130. In certain embodiments, the plunger rod 140 and plunger tip 150 may be formed from or comprise a polymeric material such as, but not limited to, an acrylonitrile-butadiene-styrene polymer, a polycarbonate, a polypropylene, a nylon, and/or a cyclo-olefin polymer or copolymer. Plunger rods 140 and plunger tips 150 formed from other suitable materials are also within the scope of this disclosure.

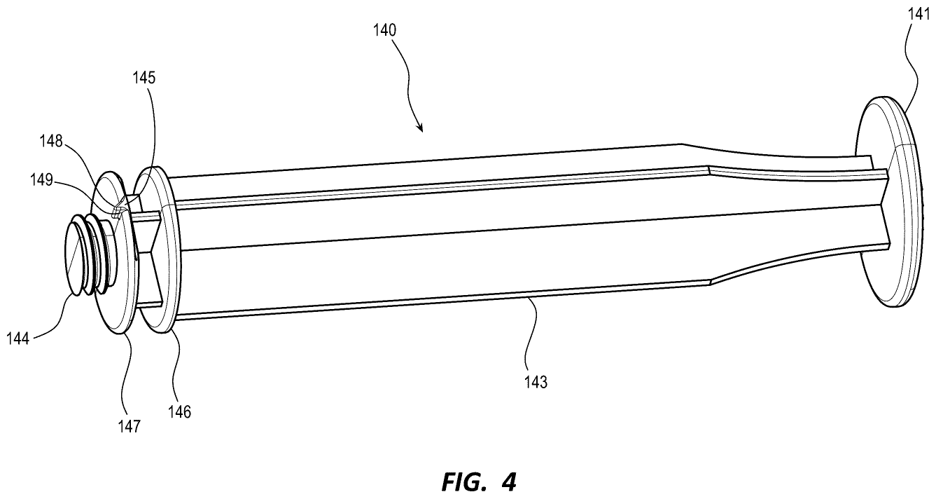

FIG. 4 is a perspective view of an embodiment of the plunger rod 140. As illustrated in FIG. 4, the plunger rod 140 can have an elongated shape with a thumb tab 141 disposed at a proximal end and oriented transverse to a longitudinal axis of the plunger rod 140. The thumb tab 141 can be configured for a user to apply a distally or proximally directed force to the plunger member 130 to displace the plunger member 130 relative to the barrel 110. A plurality of radially extending fins or ribs 143 may extend along at least a portion of a length of the plunger rod 140 between the thumb tab 141 and a proximal flange 146. In the illustrated embodiment, the number of fins is four. In other embodiments, the number of fins 143 may be two, three, five, six, or more. The proximal flange 146 may be disposed adjacent or proximate of a distal end of the plunger rod 140. The proximal flange 146 can be configured to center the plunger rod 140 within the barrel 110 as the plunger member 130 is longitudinally displaced.

A distal flange 147 can be positioned distally of the proximal flange 146. A rotational locking detent 145 may be disposed on a distally facing surface of the distal flange 147. The locking detent 145 can include an angled face 148 and a substantially normal or perpendicular face 149. The angled face 148 may include an angle ranging between 30 degrees and about 60 degrees, and may include an angle of about 45 degrees relative to the distally facing surface. The perpendicular face 149 can include an angle of about 90 degrees relative to the distally facing surface. The locking detent 145 can be configured to mate with a feature of the plunger tip 150 as explained below. A plunger rod tip 144 can extend distally from the distal flange 147. In the illustrated embodiment, the plunger rod tip 144 may include an external thread. The external thread can be a single start thread having a pitch ranging between about 1 millimeters and 2.5 millimeters, and having a pitch of about 1.5 millimeters. In some embodiments, the external thread can be a single start thread having a pitch ranging between about 1.016 millimeters and about 2.032 millimeters, and having a pitch of about 1.5875 millimeters. The plunger rod tip 144 can allow rotational coupling of the plunger tip 150 to the plunger rod 140. In other embodiments, the plunger rod tip 144 may include any other suitable type of coupling member to facilitate coupling of the plunger rod 140 with the plunger tip 150. For example, the plunger rod tip 144 may include a snap fit, a press fit, a friction fit, a bayonet lock, etc.

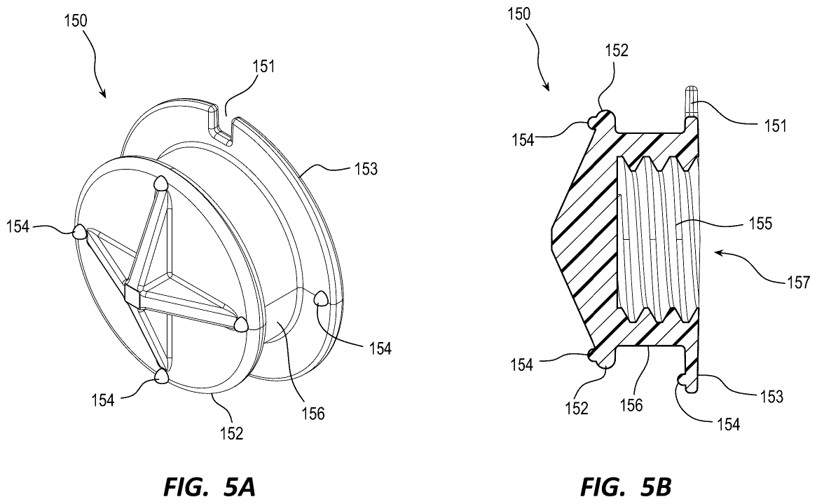

FIG. 5A is a perspective view of an embodiment of the plunger tip 150 and FIG. 5B is a cross-sectional side view of the plunger tip 150 of FIG. 5A. As shown in FIGS. 5A and 5B, the plunger tip 150 can include a distal flange 152 and a proximal flange 153 with a medial portion 156 disposed therebetween. A diameter of the medial portion 156 can be smaller than diameters of the distal flange 152 and the proximal flange 153. The diameter of the distal flange 152 may be smaller than the diameter of the proximal flange 153. A slot 151 may be disposed through the proximal flange 153. The slot 151 can be configured to engage with the locking detent 145 to restrict rotation of the plunger rod 140 in a first direction relative to the plunger tip 150 when the plunger rod 140 and the plunger tip 150 are coupled together to prevent removal of the plunger rod 140 from the plunger tip 150. A wall of the slot 151 may engage with the perpendicular face 149 when the plunger rod 140 is rotated in the first direction to prevent decoupling of the plunger rod 140 from the plunger tip 150.

The plunger tip 150 may include an internal thread 155 disposed within a bore 157 and configured to facilitate rotational coupling of the plunger tip 150 with the plunger rod 140. The internal thread can be a single start thread having a pitch ranging between about 1 millimeters and about 2.5 millimeters, and having a pitch of about 1.5 millimeters. In certain embodiments, the internal thread may be configured to threadingly engage with the external thread of the plunger rod tip 144 when the plunger rod 140 is coupled to the plunger tip 150. The single starts of the internal and external threads may be oriented such that the proximal flange 153 bottoms out or contacts the distal flange 147 when the slot 151 engages with the locking detent 145. Nubs 154 can extend distally from distally facing surfaces of the distal and/or proximal flanges 152, 153. The nubs 154 can be configured to engage with the seal member 170 to prevent rotation of the plunger tip 150 relative to the seal member 170.

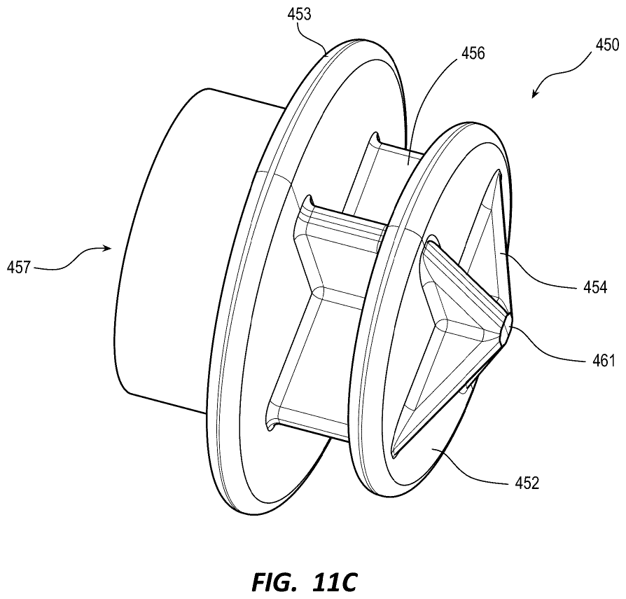

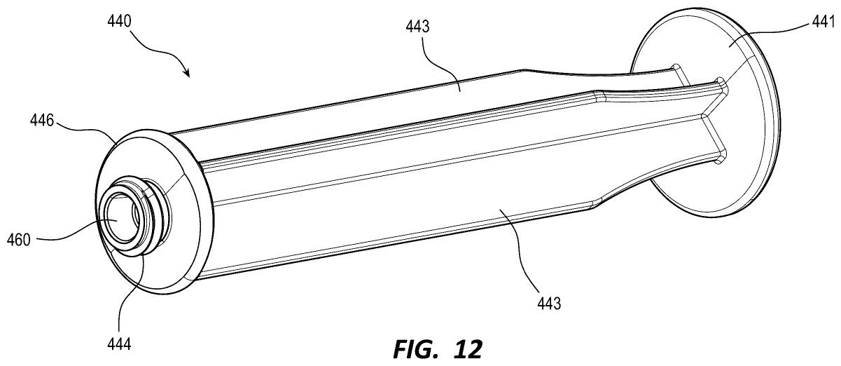

Turning ahead in the drawings, FIGS. 11-12 additional embodiments of a plunger rod 440 and a plunger tip 450 that may be used with the syringe bodies 105, the barrels 110, and/or the seal member 170. Unless otherwise noted, the plunger rod 440 and the plunger tip 450 may include any aspect of the plunger rod 140 and/or the plunger tip 150. Accordingly, unless otherwise noted, the plunger rod 440 and the plunger tip 450 may provide any of the functions and features described elsewhere in this document in relation to the plunger rod 140 and/or the plunger tip 150. For example, the plunger member 130 can comprise the plunger rod 440, the plunger tip 450 coupled to a distal end of the plunger rod 440, and a seal member 170 coupled to the plunger tip 450 wherein the plunger tip 450 is disposed between the plunger rod 440 and the seal member 170.

Turning specifically to FIG. 12, a perspective view of an embodiment of the plunger rod 440 is shown. As illustrated in FIG. 12, the plunger rod 440 can have an elongated shape with a thumb tab 441 disposed at a proximal end and oriented transverse to a longitudinal axis of the plunger rod 440. The thumb tab 441 can be configured for a user to apply a distally or proximally directed force to the plunger member 130 to displace the plunger member 130 relative to the barrel 110. A plurality of radially extending fins or ribs 443 may extend along at least a portion of a length of the plunger rod 440 between the thumb tab 441 and a distal flange 446. In the illustrated embodiment, the number of fins is four. In other embodiments, the number of fins 443 may be two, three, five, six, or more. The distal flange 446 may be disposed adjacent or proximate of a distal end of the plunger rod 440. The distal flange 446 can be configured to center the plunger rod 440 within the barrel 410 as the plunger member 130 is longitudinally displaced.

A plunger rod tip 444 can extend distally from the distal flange 446. In the illustrated embodiment, the plunger rod tip 444 may include an external thread. The external thread can be a single start thread having a pitch ranging between about 1 millimeters and 2.5 millimeters, and having a pitch of about 1.5 millimeters. The plunger rod tip 444 can allow rotational coupling of the plunger tip 450 to the plunger rod 440. In other embodiments, the plunger rod tip 444 may include any other suitable type of coupling member to facilitate coupling of the plunger rod 440 with the plunger tip 450. For example, the plunger rod tip 444 may include a snap fit, a press fit, a friction fit, a bayonet lock, etc. In some embodiments, the plunger rod tip 444 may include a recess 460 on a distal end thereof that is sized complementary to an inner rim 459 or protrusion in a bore 457 of the plunger tip 450 such that the inner rim 459 may fit at least partially within the recess 460 of the plunger rod tip 444. The inner rim 459 and the recess 460 may be configured to provide a friction lock or securement between the plunger tip 450 and the plunger rod 440. For example, the inner rim 459 may interference fit with the recess 460. This interaction of the interference fit may releasably lock the plunger rod 440 and the plunger tip 450, thereby preventing or inhibiting the plunger rod 440 and the plunger tip 450 from coming apart.

FIGS. 11A and 11C are perspective views of an embodiment of the plunger tip 450, and FIG. 11B is a cross-sectional side view of the plunger tip 450 of FIG. 11A. As shown in FIGS. 11A-11C, the plunger tip 450 can include a distal flange 452 and a proximal flange 453 with a medial portion 456 disposed therebetween. The medial portion 456 may include a plurality of radially extending fins or ribs that extend along at least a portion of a length of the medial portion 456 between the distal flange 452 and a proximal flange 453. In the illustrated embodiment, the number of fins in the medial portion 456 is four. In other embodiments, the number of fins in the medial portion 456 may be two, three, five, six, or more. An outer diameter (that includes the plurality of radially extending fins) of the medial portion 456 can be smaller than diameters of the distal flange 452 and a proximal flange 453. The diameter of the distal flange 452 may be smaller than the diameter of the proximal flange 453.

The plunger tip 450 may include an internal thread 455 disposed within a bore 457 and configured to facilitate rotational coupling of the plunger tip 450 with the plunger rod 440. The internal thread can be a single start thread having a pitch ranging between about 1 millimeters and about 2.5 millimeters, and having a pitch of about 1.5 millimeters. In certain embodiments, the internal thread may be configured to threadingly engage with the external thread of the plunger rod tip 444 when the plunger rod 440 is coupled to the plunger tip 450. The plunger tip 450 also may include an inner rim 459 or protrusion disposed in the bore 457 opposite to the opening of the bore 457. The inner rim 459 may fit within the recess 460 of the plunger rod tip 444 such that at least a portion of the plunger rod tip 444 fits between the inner rim 459 and the internal wall including the internal thread 455 of the plunger tip 455 to form friction lock between the plunger tip 450 and the plunger rod tip 444.

Ribs 454 can extend distally from distally facing surfaces of the distal flange 452. The ribs 454 can be configured to engage with the seal member 170 to support the seal member 170 when the seal member 170 is under pressure. In some embodiments, the ribs 454 are aligned with the fins of the medial portion 456. For example, in the illustrated embodiment, the number of fins in the medial portion 456 and the number of ribs 454 on the distally facing surface of the distal flange 452 is four, with the fins of the medial portion 456 and the ribs 454 being generally aligned with one another. In other embodiments, the number of fins in the medial portion 456 and the number of ribs 454 on the distally facing surface of the distal flange 452 may be two, three, five, six, or more. In other embodiments, the fins of the medial portion 456 and the ribs 454 may be misaligned. In some embodiments, the ribs 454 may connect with one another at the axis or center point 461 of the plunger tip 450. The ribs 454 may angle or taper towards the distally facing surface of the distal flange 452 from the axis or center point 461 towards an outer periphery of the distal flange 452.

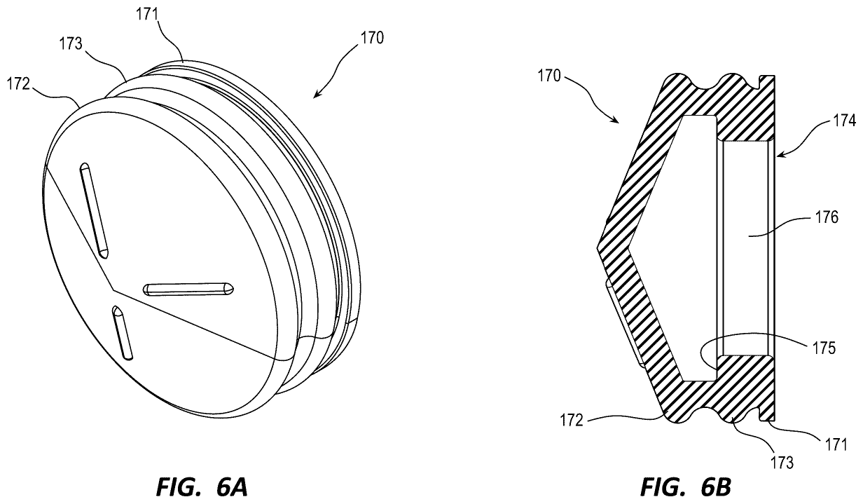

As shown, in FIGS. 6A and 6B, the seal member 170 can comprise a proximal ridge or seal ring 171 disposed around at least a portion of a circumference of the seal member 170 at or adjacent a proximal end of the seal member 170. The seal member 170 can also comprise a distal ridge 172 disposed around at least a portion of the circumference of the seal member 170 at or adjacent a distal end of the seal member 170. Stated another way, the distal ridge 172 may be disposed distal of the proximal ridge 171. The seal member 170 can further comprise a medial ridge 173 disposed around at least a portion of the circumference of the seal member 170 at a position between each of the proximal ridge 171 and the distal ridge 172. In certain embodiments, the seal member 170 may comprise one ridge, two ridges, three ridges, four ridges, five ridges, six ridges, or another suitable number of ridges. The seal member 170 may comprise a cavity 174 including a shoulder 175 and a proximal portion 176 disposed between the shoulder 175 and a proximal opening of the cavity 174. The cavity 174 can receive the plunger tip 150 wherein the distal flange 147 is disposed distal of the shoulder 175, as shown in FIG. 2. The proximal portion 176 can circumferentially surround the medial portion 156 of the plunger tip 150. A diameter of the proximal portion 176 may be smaller than the diameter of the distal flange 147 wherein the plunger tip 150 is prevented or restricted from decoupling from the seal member 170. Similarly, the cavity 174 can receive the plunger tip 450 wherein the distal flange 446 is disposed distal of the shoulder 175. The proximal portion 176 can circumferentially surround the medial portion 456 of the plunger tip 450. A diameter of the proximal portion 176 may be smaller than the diameter of the distal flange 446 wherein the plunger tip 450 is prevented or restricted from decoupling from the seal member 170.

The seal member 170 may be formed from a polymeric material. In some embodiments, the seal member 170 may be formed from silicone or poly-siloxane. In some other embodiments, the seal member 170 may be formed from a silicone-free material or poly-siloxane-free material. For example, the seal member 170 may be formed from a thermoplastic elastomer, such as chlorobutyl rubber, bromobutyl rubber, or neoprene. Seal members 170 formed from other suitable materials are also within the scope of this disclosure.

In various embodiments, the proximal ridge 171, the medial ridge 173, and/or the distal ridge 172 may exert a radial force on an interior surface of a first wall 111 of the barrel 110, as shown in FIG. 2. Stated another way, the proximal ridge 171, the distal ridge 172, and/or the medial ridge 173 may be configured to be compressed against the interior surface of the first wall 111 of the barrel 110. Additionally, the proximal ridge 171, the distal ridge 172, and/or the medial ridge 173 may slidably seal against the interior surface of the first wall 111 of the barrel 110. In various embodiments, each of the proximal ridge 171, the distal ridge 172, and/or the medial ridge 173 may exert substantially equal radial forces on the interior surface of the first wall 111 of the barrel 110.

In certain embodiments, the proximal ridge 171, the distal ridge 172, and/or the medial ridge 173 may be configured such that these ridges 171, 172, 173 are compressed within the barrel 110 to form a seal between an outer surface of the seal member 170 and the inner surface of the first wall 111 of the barrel 110. In some embodiments, the compressed portions of the seal member 170 may exert a radial outward force on the inner surface of the first wall 111 of the barrel 110 and may conform to the first wall 111 of the barrel 110. This sealing force may be configured to contain pressured fluids within the barrel and thus the seal member 170 may create a pressure seal when compressed within the barrel 110. In other words, in some embodiments, the proximal ridge 171, the distal ridge 172, and/or the medial ridge 173 may form a pressure seal against the inner surface of the first wall 111 of the barrel 110, analogous to an O-ring, for instance.

Kits comprising syringe systems or syringe bodies at least partially pre-loaded with an injectable material are disclosed herein. In some embodiments, the pre-loaded syringe system may comprise a barrel at least partially loaded with the injection material and a plunger in communication with an interior surface of the barrel. In some other embodiments, the pre-loaded syringe body may comprise a barrel at least partially loaded with the injection material. In certain embodiments, the injection material of the kit may be dry. In certain other embodiments, the injection material of the kit may be suspended in a liquid such as, but not limited to, a carrier liquid comprising saline.

In some embodiments, the kit and/or the components of the kit may be configured to be shelf stable for at least six months, for at least one year, for at least two years, for at least three years, or for another suitable period of time. In certain embodiments, the syringe system or syringe body of the kit may comprise sterilization compatible materials and/or irradiation compatible materials.

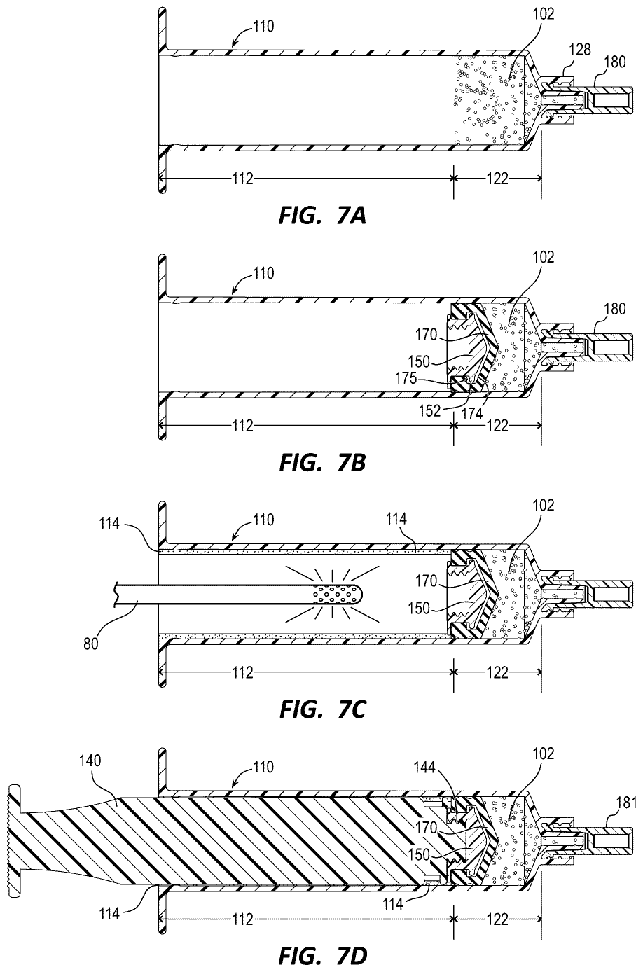

Methods of manufacture of the syringe system 100 are also disclosed herein. FIGS. 7A-7D depict an embodiment of a method of manufacture of the syringe system 100 wherein a portion of the interior surface of the barrel 110 is lubricated. While the plunger member 130 including the plunger rod 140 and the plunger tip 150 are shown in FIGS. 7A-7D, a plunger member 130 include the plunger 440 and the plunger tip 450 may be similarly used in methods of manufacture of the syringe system 100. In some embodiments, the methods may comprise obtaining a syringe body 105. As depicted in FIG. 7A, the methods may further comprise at least partially loading the barrel 110 of the syringe body 105 with the injectable material 102 wherein the injectable material 102 is disposed within the non-lubricated portion 122. In some embodiments, the injectable material 102 may comprise dry particles. A vented cap 180 may be coupled to the inlet/outlet port 128.

In some embodiments, the methods may also comprise coupling the plunger tip 150 with the seal member 170 by inserting the distal flange 152 into the cavity 174 such that the distal flange 152 is disposed distally of the shoulder 175. The assembly of the plunger tip 150 and the seal member 170 can be inserted into the barrel 110 and positioned at or adjacent the non-lubricated portion 122 as shown in FIG. 7B. The assembly of the plunger tip 150 and the seal member 170 can define a separation of the lubricated portion 112 and the non-lubricated portion 122 wherein the lubricated portion 112 is proximal and the non-lubricated portion 122 is distal of the assembly. The vented cap 180 may allow air within the barrel 110 to be vented out of the barrel 110 while maintaining the injectable material 102 within the barrel 110.

In certain embodiments, the methods may also comprise applying the lubricant 114 on at least a portion of an interior surface of a portion of the first wall 111 of the barrel 110. As shown in FIG. 7C, a lubricant applicator 80 may be used to apply the lubricant 114 to the interior surface of the lubricated portion 112 of the first wall 111 of the barrel 110. Other suitable methods of applying the lubricant 114 are also within the scope of this disclosure. For example, the lubricant 114 may be applied by dipping, swabbing, etc. As depicted, the assembly of the plunger tip 150 and the seal member 170 may be configured to inhibit, limit, or prevent exposure of a portion of the interior surface of the first wall 111. For example, the assembly may inhibit, limit, or prevent application of the lubricant 114 to the non-lubricated portion 122 of the barrel 110. The methods may also comprise, adhering, cross-linking, or curing the applied lubricant 114, i.e., to the interior surface of the lubricated portion 112 of the first wall 111 of the barrel 110.

As depicted in FIG. 7D, the methods may also comprise coupling the plunger rod 140 to the plunger tip 150 by engaging the external threads of the plunger rod tip 144 with the internal threads of the plunger tip 150 when the plunger rod 140 is rotated in the first direction relative to the plunger tip 150. The plunger rod 140 and the plunger tip 150 may be substantially rotationally locked together when the slot 151 engages with the locking detent 145 (not shown but previously described). In some embodiments, the vented cap 180 may be replaced by a non-vented cap 181 to prevent outflow of the injectable material 102 until the time of use.

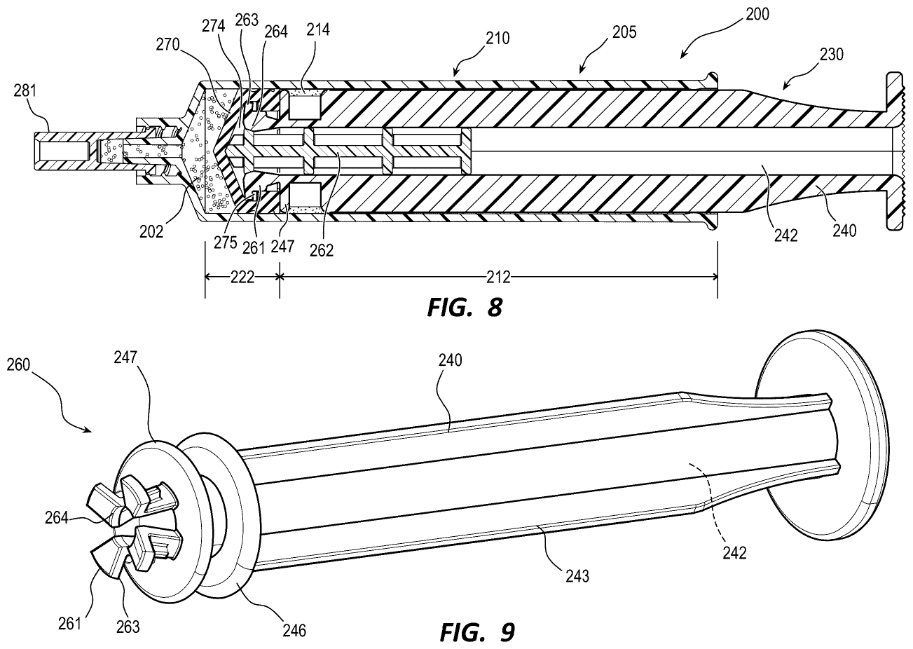

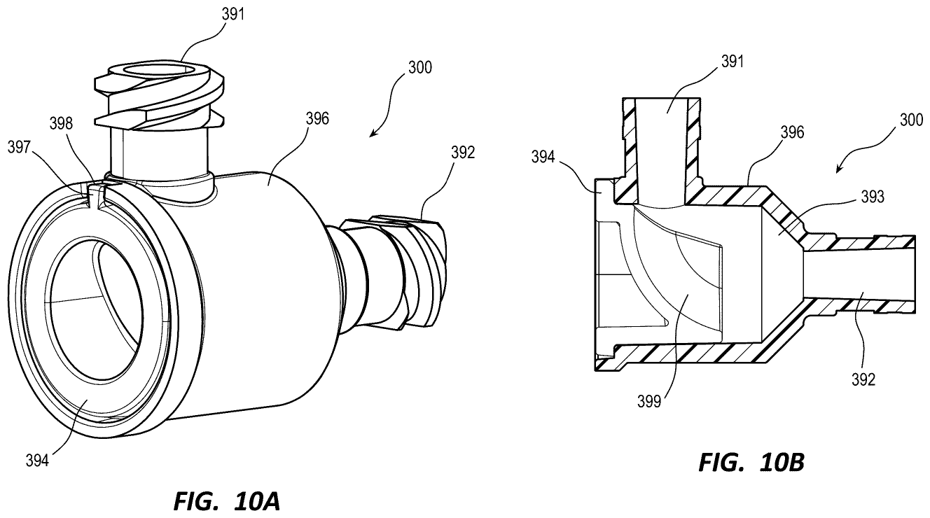

FIGS. 8 and 9 depict an embodiment of a syringe system 200 that resembles the syringe system 100 described above in certain respects. Accordingly, like features are designated with like reference numerals, with the leading digit incremented to “2.” For example, the embodiment depicted in FIGS. 8 and 9 includes a plunger rod 240 that may, in some respects, resemble the plunger rod 140 of FIG. 4. Relevant disclosure set forth above regarding similarly identified features thus may not be repeated hereafter. Moreover, specific features of the syringe system 100 and related components shown in FIGS. 1-7D may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the syringe system 200 and related components depicted in FIGS. 8 and 9. Any suitable combination of the features, and variations of the same, described with respect to the syringe system 100 and related components illustrated in FIGS. 1-7D can be employed with the syringe system 200 and related components of FIGS. 8 and 9, and vice versa. This pattern of disclosure applies equally to further embodiments depicted in subsequent figures and described hereafter, wherein the leading digits may be further incremented.

As illustrated in FIG. 8, the syringe system 200 may include a syringe body 205 comprising a barrel 210 and a plunger 230 disposed within the barrel 210. The barrel 210 may include a proximal or lubricated portion 212 and a distal or non-lubricated portion 222. A non-vented cap 281 can be coupled to a distal end of the barrel 210. An injectable material 202 may be disposed within the non-lubricated portion 222. At least a portion of an interior surface of the barrel 210 disposed in the lubricated portion 212 may be at least partially coated with a lubricant or lubricant coating 214. In contrast to the lubricated portion 212, at least a portion of the interior surface of the barrel 210 disposed in the non-lubricated portion 222 may lack the lubricant or lubricant coating 214.

As illustrated in FIGS. 8 and 9, the plunger 230 can include a plunger rod 240. The plunger rod 240 can comprise a plunger rod tip 260 disposed at a distal end. In some embodiments, the plunger rod tip 260 may be configured to couple with a seal member 270. The plunger rod tip 260 may include a plurality of anchors 261. The plunger rod tip 260 can be integrally formed with the plunger rod 240. In other embodiments, the plunger rod tip 260 may be selectively coupleable to the plunger rod 240. In the depicted embodiment, the plunger rod tip 260 has four anchors 261. In other embodiments, the plunger rod tip 260 may comprise two, three, five, or more anchors 261. The anchors 261 can be radially flexible. In a natural state, the anchors 261 may be biased radially inward as shown in FIG. 9. Each of the anchors 261 can include a foot portion 263 extending radially outward and a heel portion 264 disposed radially inward from the foot portion 263. The plunger rod 240 may include a bore 242 extending along a length of the plunger rod 240. The bore 242 can be in communication with the plunger rod tip 260 and include an opening at a proximal end of the plunger rod 240.

As illustrated in FIG. 8, when the plunger rod 240 is coupled to the seal member 270, the plunger rod tip 260 can be disposed within a cavity 274 of the seal member 270 and a plug 262 can be disposed within the bore 242. A distal end of the plug 262 may engage with the heel portion 264 of the anchors 261 to displace the anchors 261 radially outward. A proximally facing surface of the foot portion 263 of the anchors 261 may couple with a shoulder 275 of the seal member 270 to inhibit or prevent the plunger rod 240 from decoupling from the seal member 270 when a proximally directed force is applied to the plunger rod 240.

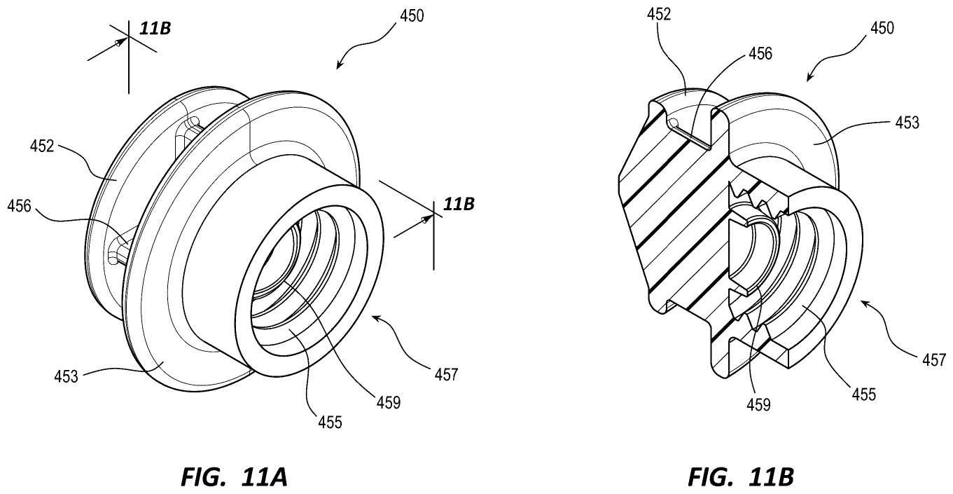

FIGS. 10A and 10B illustrate an embodiment of an injectable material container 300. The container 300 may comprise a body member 396 and a plug 394. The body member 396 can define a chamber 393 configured to hold an injectable material. A first port 391 and a second port 392 can be in communication with the chamber 393. The first and second ports 391, 392 may include fittings (e.g., female Luer lock fittings) configured for coupling of a fluid delivery device (e.g., syringe). The first and second ports 391, 392 may be disposed at a right angle to each other. In another embodiment, the first and second ports 391, 392 can be disposed at an angle ranging from about 90 degrees to about 180 degrees. The plug 394 can be coupled to the body member 396 to close an opening of the chamber 393. The plug 394 can include a radiused or curved surface 399 disposed within the chamber 393. The curved surface 399 may provide a flow path as an injectable fluid is injected through the chamber 393 to hydrate the injectable material. The plug 394 may be configured such that the flow is turbulent or non-turbulent, and can be configured to induce a type of flow that is configured to efficiently hydrate an injectable material. The plug 394 can include a radially extending tab 397 coupleable with a slot 398 in the body member 396. When the tab 397 and slot 398 are coupled, the curved surface 399 may be oriented to face toward the first and second ports 391, 392. In certain embodiments, the injectable material container 300 may include non-vented caps (not shown) coupled to the first and second ports 391, 392 to contain the injectable material in the chamber 393 until the injectable material container 300 is ready to use.

In use, the injectable material container 300 may be utilized to hydrate the injectable material disposed within the chamber 393. A method of hydrating injectable material may comprise the steps of coupling a first fluid delivery device to the first port 391 of the injectable material container 300 and coupling a second fluid delivery device to the second port 392 of the injectable material container 300. The first and/or second fluid delivery device may comprise an injectable fluid (e.g., saline and/or contrast media). The injectable fluid from the first fluid delivery device can be injected through the first port 391, through the chamber 393, through the second port 392, and into the second fluid delivery device. When the injectable fluid passes through the chamber 393, the injectable material may be displaced by the injectable fluid into the second fluid delivery device. To ensure mixing and hydrating of the injectable material with the injectable fluid, the injectable fluid and injectable material can be repeatably displaced between the first and second fluid delivery devices through the chamber 393.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of manufacture of a syringe system may include one or more of the following steps: filling a distal reservoir portion of a syringe barrel with injectable particles; positioning a seal member within the syringe barrel, wherein a distal reservoir portion of the syringe barrel is disposed distal of the seal member and a proximal reservoir portion of the syringe barrel is disposed proximal of the seal member; applying a lubricant to an internal surface of the proximal reservoir portion; and coupling a syringe plunger to the seal member, wherein the syringe plunger comprises: a plunger rod; and a plunger tip disposed at a distal end of the plunger rod. Other steps are also contemplated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

“Fluid” is used in its broadest sense, to refer to any fluid, including both liquids and gases as well as solutions, compounds, suspensions, etc., which generally behave as fluids.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the proximal end of a syringe system is defined as the end closest to the practitioner during utilization of the syringe system. The distal end is the end opposite the proximal end, along the longitudinal direction of the syringe system.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A syringe plunger, comprising:
a plunger rod defining a proximal end and a distal end;
a plunger tip disposed at the distal end; and
a seal member coupled to the plunger tip;
wherein the plunger tip is configured to prevent movement of the plunger rod relative to the seal member.

2. The syringe plunger of claim 1, wherein the plunger tip comprises:
an internal thread configured to threadingly engage with an external thread disposed at the distal end of the plunger rod when the plunger tip is coupled to the plunger rod; and
a proximal flange comprising an anti-rotation member configured to prevent rotation of the plunger rod relative to the plunger tip when is coupled to the plunger rod, wherein the anti-rotation member comprises a slot disposed through the proximal flange and configured to engage with a detent disposed on a distal flange of the plunger rod.

3. The syringe plunger of claim 2, wherein the detent comprises a first face and a second face, wherein the first face comprises an acute angle relative to a distal surface of the distal flange, wherein the second face comprises a perpendicular angle relative to the distal surface, and wherein the first face is configured to allow rotation of the plunger rod relative to the plunger tip in a first direction and the second face is configured to prevent rotation of the plunger rod relative to the plunger tip in a second direction when the detent is engaged with the slot.

4. The syringe plunger of claim 2, wherein the internal thread and the external thread are a single start threads, and wherein a single start of the internal thread and a single start of the external thread are configured to position the slot into engagement with the detent when the internal thread and the external thread are fully engaged.

5. The syringe plunger of claim 1, wherein the plunger tip comprises a plurality of nubs configured to engage with an interior surface of the seal member to prevent rotation of the seal member relative to the plunger tip.

6. The syringe plunger of claim 1, wherein the plunger tip comprises:
a plurality of anchors coupled to and extending distally from the distal end of the plunger rod,
wherein each of the plurality of anchors is biased radially inward.

7. The syringe plunger of claim 6, wherein the plunger rod further comprises:
a plug disposed within a bore of the plunger rod,
wherein the plug is configured to displace each of the plurality of anchors radially outward to prevent the seal member from uncoupling from the distal end of the plunger rod when the plunger rod is displaced proximally.

8. The syringe plunger of claim 1, wherein the plunger tip includes a distal flange, a proximal flange, and a medial portion disposed between the distal flange and the proximal flange, the medial portion including a plurality of radially extending fins or ribs.

9. The syringe plunger of claim 1, wherein the plunger tip includes a plurality of ribs extending distally from a distal facing surface of a distal flange of the plunger tip, the plurality of ribs angling or tapering from a center or axis of the plunger tip towards an outer periphery of the distal flange.

10. The syringe plunger of claim 1, wherein:
the plunger tip includes a bore having an internal thread and an inner rim disposed therein; and
a distal end of the plunger rod includes a recess shaped complementary to the inner rim and positioned to receive at least a portion of the inner rim therein when the plunger tip is threadingly coupled to the distal end of the plunger rod.

11. A syringe system, comprising:
a plunger member comprising:
a plunger rod defining a proximal end and a distal end;
a plunger tip disposed at the distal end; and
a seal member coupled to the plunger tip;
wherein the plunger tip is configured to prevent movement of the plunger rod relative to the seal member.

12. The syringe system of claim 11, wherein the plunger tip comprises:
an internal thread configured to threadingly engage with an external thread disposed at the distal end of the plunger rod when the plunger tip is coupled to the plunger rod; and
a proximal flange comprising an anti-rotation member configured to prevent rotation of the plunger rod relative to the plunger tip when is coupled to the plunger rod, wherein the anti-rotation member comprises a slot disposed through the proximal flange and configured to engage with a detent disposed on a distal flange of the plunger rod.

13. The syringe system of claim 12, wherein the detent comprises a first face and a second face, wherein the first face comprises an acute angle relative to a distal surface of the distal flange, wherein the second face comprises a perpendicular angle relative to the distal surface, and wherein the first face is configured to allow rotation of the plunger rod relative to the plunger tip in a first direction and the second face is configured to prevent rotation of the plunger rod relative to the plunger tip in a second direction when the detent is engaged with the slot.

14. The syringe system of claim 12, wherein the internal thread and the external thread are a single start threads, and wherein a single start of the internal thread and a single start of the external thread are configured to position the slot into engagement with the detent when the internal thread and the external thread are fully engaged.

15. The syringe system of claim 11, wherein the plunger tip comprises a plurality of nubs configured to engage with an interior surface of the seal member to prevent rotation of the seal member relative to the plunger tip.

16. The syringe system of claim 11, wherein the plunger tip comprises:
a plurality of anchors coupled to and extending distally from the distal end of the plunger rod,
wherein each of the plurality of anchors is biased radially inward.

17. The syringe system of claim 16, wherein the plunger further comprises:
a plug disposed within a bore of the plunger rod,
wherein the plug is configured to displace each of the plurality of anchors radially outward to prevent the seal member from uncoupling from the distal end of the plunger rod when the plunger rod is displaced proximally.

18. The syringe system of claim 11, wherein the plunger tip includes a distal flange, a proximal flange, and a medial portion disposed between the distal flange and the proximal flange, the medial portion including a plurality of radially extending fins or ribs.

19. The syringe system of claim 11, wherein the plunger tip includes a plurality of ribs extending distally from a distal facing surface of a distal flange of the plunger tip, the plurality of ribs angling or tapering from a center or axis of the plunger tip towards an outer periphery of the distal flange.

20. The syringe system of claim 11, wherein:
the plunger tip includes a bore having an internal thread and an inner rim disposed therein; and
a distal end of the plunger rod includes a recess shaped complementary to the inner rim and positioned to receive at least a portion of the inner rim therein when the plunger tip is threadingly coupled to the distal end of the plunger rod.
